(19) United States
(12) Patent Application Publication
(10) Pub. No.: US 2014/0275679 A1
(43) Pub. Date: Sep. 18, 2014

(54) REACTOR, PROCESS, AND SYSTEM FOR THE OXIDATION OF GASEOUS STREAMS

(71) Applicant: John A. Sofranko, Princeton, NJ (US)

(72) Inventor: John A. Sofranko, Princeton, NJ (US)

(73) Assignee: Bio2Electric, LLC, Princeton, NJ (US)

(21) Appl. No.: 14/289,995

(22) Filed: May 29, 2014

Related U.S. Application Data
(63) Continuation-in-part of application No. PCT/US2012/066789, filed on Nov. 28, 2012.
(60) Provisional application No. 61/609,394, filed on Mar. 12, 2012.
(60) Provisional application No. 61/566,176, filed on Dec. 2, 2011.
(60) Provisional application No. 61/577,353, filed on Dec. 19, 2011.

(51) Int. Cl. C07C 2/84; B01J 19/08

(52) U.S. Cl. CPC C07C 2/84; B01J 19/087; B01J 2219/0803
USPC 585/310; 422/222; 422/186

(57) ABSTRACT

A reactor and process capable of concurrently producing electric power and selectively oxidizing gaseous components in a feed stream, such as hydrocarbons to unsaturated products, which are useful intermediates in the production of liquid fuels. The reactor includes an oxidation membrane, a reduction membrane, an electron barrier, and a conductor. The oxidation membrane and reduction membrane include an MIEC oxide. The electron barrier, located between the oxidation membrane and the reduction membrane, is configured to allow transmission of oxygen anions from the reduction membrane to the oxidation membrane and resist transmission of electrons from the oxidation membrane to the reduction membrane. The conductor conducts electrons from the oxidation membrane to the reduction membrane.

### CROSS REFERENCE TO RELATED APPLICATIONS

The present application is a Continuation-in-Part of PCT/US2012/066789, filed Nov. 28, 2012, which claims priority to and benefit of U.S. Provisional Patent Application No. 61/566,176, filed Dec. 2, 2011, U.S. Provisional Patent Application No. 61/577,353, filed Dec. 19, 2011, and U.S. Provisional Patent Application No. 61/609,394, filed Mar. 12, 2012, the entire disclosures of all of which are incorporated herein by reference in their entireties for all purposes.

### FIELD OF THE INVENTION

The invention relates to reactors and processes used to convert gaseous streams containing hydrocarbon gases, for example, into intermediates useful in the production of higher molecular weight products, such as liquid fuels.

### BACKGROUND OF THE INVENTION

Driven by a growth in worldwide natural gas supply, and increasing value to liquid fuels, there is considerable interest in the conversion of natural gas to liquid fuels. Much of the newly discovered natural gas is in remote areas of the world where the cost of constructing conventional gas-to-liquid (GTL) plants is uneconomical. Significant natural gas reserves are being developed from hydrocarbon resources contained in shale deposits. Over one quarter of the methane released into the atmosphere in the U.S. is emitted from landfills and manure treatment. These reserves are often far from large refining centers and require significant capital investment to bring these reserves to market.

One of the major challenges of conventional Fischer-Tropsch (FT) technologies is the production of a mixture of hydrocarbon products from the synthesis step. These products require further refining and blending to capture the value of the products, which include diesel, LPG, and naphtha. Therefore, capital intensive mega petrochemical chemical complexes are required. Conventional FT technology is not suitable for smaller gas sources that may be remote, isolated, or distributed around the world.

There is therefore a need for GTL technology that allows for implementation at smaller scale and in remote locations without a large capital investment.

### SUMMARY OF THE INVENTION

An embodiment of the invention includes a solid oxidation membrane having mixed ionic electronic conductive (MIEC) properties, the oxidation membrane comprising a material having a cubic crystal lattice structure and a chemical formula of:

A₆BO₈

wherein A is a first element, B is a second element that is different than the first element, and O is oxygen, and wherein the oxidation membrane includes an oxidation zone configured to receive a gaseous feedstream and oxidize components of the feedstream.

As used herein throughout the specification and claims, a material having “MIEC” properties means a material through which electrons and oxygen anions may be conducted.

Another embodiment of the invention includes an electrogenerative reactor. The reactor comprises an oxidation membrane comprising an MIEC oxide; a reduction membrane also containing an MIEC oxide; an electron barrier between the oxidation membrane and the reduction membrane, the electron barrier configured to allow the passage of oxygen anions from the reduction membrane to the oxidation membrane and resist the passage of electrons from the oxidation membrane to the reduction membrane; and a conductor configured to conduct electrons from the oxidation membrane to the reduction membrane.

Another embodiment of the invention includes a process comprising supplying a feedstream to the oxidation membrane of a reactor; supplying oxygen to the reduction membrane of the reactor; generating a current through a conductor of the reactor; conducting an effluent containing the intermediate from the reactor to a vessel; and converting the intermediate to a product in a vessel, wherein the product has a higher molecular weight than the intermediate.

Yet another embodiment of the present invention is a system that includes a first feed stream comprising at least one of a hydrocarbon, sulfur containing compound, nitrogen containing compound, alcohol, and carbon monoxide; a second feed stream comprising oxygen; a reactor configured to receive the first and second streams, operate within a temperature range, and produce an intermediate effluent, wherein the reactor comprises a solid oxidation membrane having MIEC properties, the oxidation membrane comprising a material having a cubic crystal lattice structure and a chemical formula of:

A₆BO₈

wherein A is a first element, B is a second element that is different than the first element, and O is oxygen; and the system also includes a vessel that receives the intermediate effluent and produces a product, the product having a higher molecular weight than the intermediate.

Other aspects and advantages of the invention will be apparent from the following detailed description wherein reference is made to the accompanying drawings.

### BRIEF DESCRIPTION OF THE FIGURES

In order that the invention may be more fully understood, the following figures are provided by way of illustration, in which:

FIG. 1 is a simplified schematic block diagram of a reactor according to an embodiment of the present invention;

FIG. 2 is a simplified schematic block diagram of a reactor according to another embodiment of the present invention;

FIG. 3 is a simplified schematic block diagram of a process according to yet another embodiment of the present invention;

FIG. 4 is the open circuit potential over time for a reactor configured similar to FIG. 1 which includes an anode catalyst according to yet another embodiment of the present invention; and

FIG. 5 is the current density vs. the voltage exhibited for a reactor configured similar to FIG. 1, which includes an anode catalyst according to yet another embodiment of the present invention, and fed a stream of methane with and without steam.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

According to one embodiment of the invention, a reactor is provided that may concurrently produce an intermediate and electric power. The reactor comprises an oxidation membrane containing an oxidizing catalyst. The oxidizing catalyst may oxidize a number of compounds including hydrocarbons, such as methane, that are supplied to the oxidation membrane to form unsaturated intermediates, such as ethylene. The oxidizing catalyst preferably comprises an MIEC oxide which enables the transmission of oxygen ions and electrons across the oxidation membrane. The reactor further comprises a reduction membrane also comprising an MIEC oxide. Oxygen is preferably provided by supplying air to the reduction membrane. The oxygen reducing catalyst converts the oxygen to oxygen anion which is transmitted across the reduction membrane to the oxidation membrane. Electrons may also travel across the reduction membrane; however, the reactor further comprises an electrolytic material in the form of an oxygen ion conducting membrane between the oxidation and reduction membranes. The electrolytic material allows transmission of oxygen anions from the reduction membrane to the oxidation membrane while resisting the transmission of electrons from the oxidation membrane to the reduction membrane. The electron barrier may also be configured to resist the passage of monovalent or molecular oxygen from the reduction membrane to the oxidation membrane. The reactor further comprises a conductor attached to the oxidation and reduction membranes to circumvent the electron barrier and allow the electrons to travel from the oxidation membrane to the reduction membrane, thereby generating a current through the conductor from which electric power may be drawn.

According to another embodiment of the invention, a process is provided that comprises providing a gaseous stream to the oxidation membrane of the reactor as described above to produce an intermediate, supplying oxygen to the reduction membrane of the reactor, generating a current through a conductor of the reactor, conducting an effluent containing the intermediate from the reactor to a vessel, and converting the intermediate to a product in the vessel, wherein the product has a higher molecular weight than the intermediate. The product may be, for example, a liquid hydrocarbon, and the gaseous stream may include methane. Thus, the methane is converted to larger hydrocarbons in a two-step process. In the first step hydrocarbons are oxidized from a reduced form to an oxidized form either by homologation of carbon-carbon bonds or by increasing the degrees of unsaturation in the product. In a second step, the product from the first step is converted to higher molecular weight products by the action of a catalyst in a vessel (32) as illustrated in FIG. 3.

The reactor and process may be used to oxidize a number of compounds and is not limited to the production of higher molecular weight products such as liquid fuels. Additional components in the gaseous stream fed to a reactor according to the present invention may include hydrogen or compounds such as saturated or unsaturated hydrocarbons, such as methane or aromatics, sulfur containing compounds, such as hydrogen sulfide or sulfur oxides, nitrogen containing compounds, such as nitrogen oxides or ammonia, alcohols, and carbon monoxide.

The invention may be used to maximize the conversion of hydrocarbons to more valuable hydrocarbons while some lower value products, such as carbon dioxide and carbon monoxide, may also be formed. Additional components of the effluent may include oxygenated hydrocarbons, such as alcohols, which are formed in preference to the production of carbon monoxide or carbon dioxide, resulting in an effluent from the reactor with a higher molar concentration of oxygenated hydrocarbons than carbon monoxide or carbon dioxide. The lower value products may be separated, for example, in a separator (24) as illustrated in FIG. 3, from the product stream such that any unconverted, or under-converted feed, may be recycled (25) to the reactor (22). Alternatively, the intermediate product produced in the reactor, if desirable may be separated directly from the reaction stream by known means. For example, ethane and propane may be converted to ethane and propylene and used for commercial application.

According to yet another embodiment of the present invention, a process either has an overall reaction in which the Gibbs free energy change is positive, ΔG≧0, or enhances the rate of oxygen anion mobility in the reactor. Referring to FIG. 2, power (9) is applied through the conductor (17) to a cathode plate (16), which promotes the reduction of oxygen to oxygen anion in the cathode membrane (11). Oxygen anion moves through the electron barrier (13), which is intimately associated with the selective oxidation catalyst in the oxidation membrane (10). The oxidation of of the components in the feed stream occurs in the oxidation membrane and electrons are conducted through an anode plate (15) to complete the power circuit. In this oxygen pumping embodiment the effective pressure differential of oxygen between the reduction membrane and oxidation membrane is increased, thus increasing the rate of oxygen anion transfer through the electron barrier.

The reaction vessel may include an acidic catalyst to facilitate the conversion of intermediates to a product. Optionally, the process may further comprise a second conducting step to conduct the product and at least one of unconverted intermediates, under-converted intermediates, carbon monoxide, and carbon dioxide from the vessel to a separator and separating the product in the separator from the at least one of unconverted intermediates, under-converted intermediates, carbon monoxide, and carbon dioxide. Upon separation, the process may further comprise recycling at least one of the unconverted intermediates and under-converted intermediates back to the reactor.

The reactor, described herein, will have lower capital cost and provides an advantage over traditional FT technology that is better suited for very large applications. The invention may utilize Oxidative Coupling of Methane (OCM) which is better suited for remote applications because the product can be primarily one fungible liquid fuel rather than a mixture of many hydrocarbon products as seen with FT GTL technology. The fundamental reaction of OCM to olefins is the interaction of a reactive metal oxide with methane to produce a gas phase methyl radical.

OCM reactions can be run in two different reactor configurations: redox and catalytic. In a redox mode, methane is reacted in a circulating bed reactor to form higher hydrocarbon products and the metal oxide catalyst is reduced to a non-active state. This non-active catalyst is then reactivated by air or oxygen in a second process step. The complexity of this set-up presents obstacles to the scale-up of this type of reactor system. In a catalytic mode, oxygen is used as the oxidant to prevent the need for regeneration; however, exotic chemical reactor designs, such as a thin-bed reactor need to be employed to remove significant heat generated by the reaction. In addition, the yields to desirable olefin products are lower in the catalytic mode than the redox mode. It has also been observed that there is a relationship in OCM reactions between methane conversion and C₂⁺ selectivity. Thus, at 25% methane conversion typically ˜75% C₂⁺ selectivity is observed, and at 10% conversion ˜90% C₂⁺ selectivity is reported. This has led to the speculation that there is a theoretical limit on the per-pass yield for the OCM reaction.

Surface-exposed lattice oxygen is the active species in abstracting the first hydrogen atom from methane to form methyl radicals. Catalyst membrane surfaces fed dissociated oxygen have the potential to act more selectively in methane coupling than gaseous O₂. In general, higher selectivities to C₂⁺ products are formed when catalytic materials are integrated in a catalytic membrane reactor versus the same catalyst run in a fixed bed system. This demonstrates that lattice oxygen moving to the surface of the catalyst leads to more selective OCM.

Membrane reactors used in the various embodiments of the present invention may include an MIEC dense metal oxide ceramic membrane. The ceramic material must not be permeable to oxygen molecules, but rather efficiently promote the ionic conductance of oxygen anion, O⁻², through the material, in a similar way that solid oxide fuel cell (SOFC) electrolyte materials, such as yttria stabilized zirconia (YSZ), transport oxygen ions. To be effective as an MIEC material, however, the membrane must also be electronically conductive in order to balance the charge on both sides of the membrane. In many systems, the selectivity to C₂⁺ products is higher (over 90%) when the OCM reaction is conducted over the identical catalyst system in a membrane reactor configuration versus a fixed bed reaction with co-fed oxygen. Thus, lattice oxygen is more selective for OCM than oxygen adsorbed from the gas phase. However, MIEC membrane reactors are associated with the following challenges:

High temperatures, >750° C., required to achieve adequate oxygen anion transfer rates;

Differential thermal expansion rates of membrane components;

Effective sealing of membranes operated at high temperature;

Large oxygen partial pressure gradient across the membrane which causes phase instability;

Lack of effective MIEC materials that are also selective catalytic material for OCM reactions; and

Managing a significant heat of reaction and maintaining and effective reactor temperature.

The disadvantages encountered with OCM reactions described above can be overcome via the use of lattice oxygen and strategic integration of heat and power, by implementing the reactor of the present invention.

The present invention includes electrogenerative reactors which are capable of co-generating electricity and useful products. For example, in the embodiment in FIG. 2, the unit (9) instead of being used to apply power may instead be a load from which power may be withdrawn. The electrogenerative process couples specifically designed electrochemical reactions at the two electrodes to form desired products. Compared to conventional electrolytic and heterogeneous catalysis processes, an electrogenerative system produces electric power as a byproduct. As a result, the need for an external power supply can be avoided in most cases. In addition, an electrogenerative system has the potential to be operated under a more controllable environment than conventional catalytic processes.

The integration of an electrogenerative reactor with an oligomerization reactor yields a number of process and economic advantages that include:

Eliminating the need for costly oxygen/nitrogen separation by use of a dense phase membrane reactor to promote oxidation on one side of the membrane with oxygen moieties supplied from air on the other side of the membrane;

Producing high yield to the desired C₂⁺ products by using only the selective lattice oxygen supplied by the catalyst via transport of oxygen anion through the membrane reactor;

Reaction exothermicity is reduced because reaction energy is converted to useful electricity, thus increasing the overall energy efficiency of the process compared to other processes, such as Fischer-Tropsch synthesis;

Eliminating the need to use costly separation equipment, such as cryogenic distillation towers or membrane separators, in order to remove products, such as ethylene, from a reactor effluent that contains un-reacted hydrocarbons, such as methane;

Efficient separation of CO₂ for sequestration;

Elimination of moving parts for power generation with high overall process efficiency; and

Reducing capital costs by providing OCM reactors that may be easily installed within an existing SOFC manufacturing system.

By reducing the cost of the conversion of gaseous hydrocarbons to liquid fuels and increasing the energy efficiency, the present invention has useful commercial utility, even at scales less than 10,000 BL/D.

The present invention, surprisingly, is also capable of converting various forms of sulfur containing natural gas which includes, but is not limited to biogas, shale gas, associated gas from oil & gas production, coal gas, or any other form of methane containing gas that also contains some form of sulfur, either organic or inorganic sulfur, to higher hydrocarbons. The oxidation of H₂S contained in the natural gas into SO₂ and SO₃ has been found to be synergistically beneficial for CO₂ sequestration, selectivity to C²⁺ products, and catalyst life. Generally, all sulfur in the feed provided to a reactor of the present invention is converted to SO₂, SO₃, or a mixture of the two sulfur gases.

The present invention is useful for the conversion of methane, or methane containing gases such as natural gas, to higher molecular weight molecules, which includes liquid fuels. In addition to methane as a feed for the method of this invention, other higher molecular weight hydrocarbons may be employed as feeds in accordance with the formula:

$$z {C}_{n} {H}_{2 n + 2 - 2 β} + \frac{( z - 1 + δ )}{2} {O}_{2} -> {C}_{( z \times n )} {H}_{2 ( z \times n ) + 2 - 2 β - 2 δ} + ( z - 1 + δ ) {H}_{2} O$$

where z=the number of reacting molecules; n=the number of atomic units in the reacting molecule; β=the degree of unsaturation where the value is zero for single bonds, one for double bonds and molecular rings, and two for triple bonds; and δ=the change in the degree of unsaturation.

Referring to FIG. 2, oxidative conversion takes place in an oxidation membrane (10), whereby the oxygen moiety is supplied primarily via oxygen ion transport though an electron barrier (13), that is in intimate contact with a selective catalytic material within the oxidation membrane (10) that has mixed ionic-electronic conductivity properties. The oxygen ion is produced in a reduction membrane (11) by the electrochemical reduction of oxygen in a gas (12) by using a cathode plate (16). Electrons spontaneously flow from cathode to anode membrane because of the half-cell reactions, such that the sum of these half-cell reactions yields a net production of energy:

$$z {C}_{n} {H}_{2 n + 2 - 2 β} -> {C}_{( z \times n )} {H}_{2 ( z \times n ) + 2 - 2 β - 2 δ} + 2 ( z - 1 + δ ) {H}^{+} + 2 ( z - 1 + δ ) {e}^{-}$$

$$\frac{( z - 1 - δ )}{2} {O}_{2} + 2 ( z - 1 + δ ) {e}^{-} -> ( z - 1 + δ ) {O}^{2 -} \\ ( z - 1 + δ ) {O}^{2 -} + 2 ( z - 1 + δ ) {H}^{+} -> ( z - 1 + δ ) {H}_{2} O$$

The open circuit potential for these systems can be estimated by the Nernst equation. Where by convention, when the overall reactions have negative Gibbs free energy, ΔG, the electrochemical system is thermodynamically allowed and electrons pass spontaneously from anode membrane to cathode membrane through a conductor (17). The maximum amount of electric work, Wₑ₁, that could be done by this electrogenerative reactor is thus:

Wₑ₁=ΔG=−nFE

where F is Faraday's constant, E is the reversible open circuit maximum potential and n is the number of electrons transferred. In this system, n of the Nernst equation is also the same as the term (z−1+δ) in the previous discussion;

Wₑ₁=ΔG=(z−1+δ)FE

In one embodiment of the invention, the feed (14) to the electrogenerative reactor may be, for example, any hydrocarbon from methane to C₂₄, or mixtures thereof. The maximum open circuit potentials of this invention for various reactions are shown in Table 1, as calculated using the Nernst Equation using literature values for reaction conversions at reaction temperature. Because the reaction mechanism for OCM involves gas phase methyl radicals, the actual open circuit potential for the electrogenerative reactor when converting methane as feed will likely be between the open circuit potential for the OCM to ethylene and water and the open circuit potential for the formation of water from hydrogen and oxygen. However, no mechanism is implied or required in the present invention in order to convert hydrocarbons to more oxidized hydrocarbons, as described herein.

TABLE 1

| | e-per | | | Open Circuit | |
|---|---|---|---|---|---|
| | Mole | ΔG, kJ/mole | | Potential, V | |
| Reaction | Product | 973 K | 1073 K | 973 K | 1073 K |
| 2CH₄ + ½O₂ --> CH₃CH₃ + H₂O | 2 | −121.68 | −116.24 | 0.63 | 0.60 |
| 2CH₄ + O₂ --> CH₂CH₂ + 2H₂O | 4 | −304.22 | −306.83 | 0.79 | 0.80 |
| CH₃CH₃ + ½O₂ --> CH₂CH₂ + H₂O | 2 | −182.54 | −190.43 | 0.95 | 0.99 |
| H₂ + ½O₂ --> H₂0 | 2 | −47.51 | −45.66 | 1.03 | 0.99 |

While the maximum open circuit potential is defined by Wₑ₁, the actual reactor cell potential is typically less than the ideal potential due to several types of irreversible energy losses which may include cell polarization, overpotential and related energy losses at the electrodes that stem from the activation energy of the electrochemical reactions at the electrodes, ohmic losses caused by resistance in the electrodes and electrolytes, mass-transport losses due to finite mass transport limitation rates of electro-reactants, and other irreversible energy losses of the system. The efficiency, ε, of the electrogenerative reactor described in the present invention will be:

$$ɛ = \frac{Electrical Energy Produced}{Δ G at Reaction Conditions}$$

The electrical efficiency of the electrogenerative reactor will typically be within the range of 5% to 99%, and preferably within the range of 45% to 99%.

The reaction temperature for the electrogenerative reactor must be sufficiently high enough to promote efficient ionic conductivity of oxygen anion through all membrane components of the electrogenerative cell, which includes the electrolyte membrane, cathodic mixed conductive catalyst and anodic mixed conductive catalyst. Typically oxygen anion is conducted through solid oxide materials in the range of 400° to 1,200° C., and for the present invention is preferred to be within the range of about 400° to about 1,000° C.

It is advantageous to have effective three phase contact between the catalyst materials, both anode and cathode, with the electrolyte material and the reactants. The catalyst materials may be porous, or dense, in so much as effective mass transport of reactants and electrical contact is maintained. The contact time of the feed hydrocarbons with the anode catalyst in the anode membrane, or the feed oxidant, typically air, with the cathode catalyst in the cathode membrane can be 0.01 seconds to 60 seconds, when calculated at reaction conditions of temperature and pressure. More typically, the anode and cathode catalytic contact time will be in the range of 0.1 to 20 seconds. The reaction contact times are optimized to produce the highest cell efficiency, ε, and yield of the desired product of oxidation.

The reaction pressure in the anode membrane and cathode membrane will be optimized to produce the highest cell electrochemical efficiency and yield of the desired oxidation products. Typical pressures of operation are between 0.1 and 20 atmospheres and more preferably between 1 and 15 atmospheres. The pressure may be the same, or different on the anode and cathode membranes of the reactor in so much as appropriate cell designs allow for safe operation with pressure differences between the membranes.

In a preferred embodiment of the present invention, the reduction membrane of the reactor is separated from the oxidation membrane of the reactor by intimate contact with an electrolytic membrane which operates as an electron barrier. The electrolytic membrane has very low gas diffusion, or permeation, such that it forms an effective gas separator between the cathode membrane and anode membrane. The electrolytic membrane promotes the transport of oxygen anion from the reduction membrane to the oxidation membrane. It functions primarily as an anion conductor and has low electronic conductivity, thereby forcing the electron flow from the oxidation membrane to the reduction membrane to occur primarily through a conductor which may be in the form of an external electronic circuit of the cell. A commonly used electrolyte material is yttrium stabilized zirconia, YSZ, with yttria levels in the range of 3 to 10% by weight. However, a broad range of electrolytes may be used for the current invention in so much as the electrolyte material has sufficient oxygen anion conductivity at the desired reaction temperature, has low gas diffusion rates for anode and cathode reactants, and has the proper mechanical properties to be used in the reactor. Other electrolytes that may be used include mixtures of Ce/Gd oxides; La, Sr, Ga, Mg oxides; and Sc, Zn oxides. However, any material may be used if in a solid state it conducts oxygen anion within the preferred temperature range of 400° to 1,000° C. and has oxygen anion conductivities within the range of 10⁻⁵ to 1 Q⁻¹cm⁻¹.

The physical shape of the reactor is not important as long as the unit can effectively contact the reactants within the catalytic membranes, have effective control of the reaction contact times, have suitable mechanical stability under reaction conditions, and can be manufactured as reasonable costs. The reactor may be similar in design and manufacturing techniques to tubular or planar SOFCs, but is not limited by these designs.

The oxidation membrane contains one or more catalysts that promote the reduction of the oxidant, typically oxygen, to oxygen anion and is in intimate contact with the electrolyte membrane and the electrode interconnects. The materials may include any material that may catalyze the reduction of oxygen, MIEC material properties, and have chemical stability towards the electrolyte. Typical materials are perovskites and may include strontium doped LaMnO₃ and mixed oxides of (La, is Sr)(Co, Fe)O₃ or any other mixed conductive oxide, such as those used in SOFC applications.

Materials used in the oxidation membrane in various embodiments of the present invention have MIEC properties. The materials also are capable of converting hydrocarbons to a more oxidized form by promoting the reactions of dehydrogenation or coupling of carbon-carbon bonds either in cyclic or acyclic manner. The anode materials useful for the current invention promote the oxidation of hydrocarbons to more oxidized hydrocarbons in preference to the formation of carbon dioxide, carbon monoxide, or solid carbon products commonly known as coke.

The materials, by nature of their MIEC property, can promote the selective oxidation of compounds in the presence of, or in the substantial absence of, oxygen. The reaction in the substantial absence of oxygen will only occur as long as the anode materials are at least partially oxidized, and thereby would also react with hydrogen to form water. In the oxidation membrane the materials, due to their MIEC property, can be reduced by the compounds in the feed stream and at the same time be reoxidized by oxygen anion. The oxygen anion is supplied to the anode materials via intimate contact, and ionic conductivity, with the electrolytic membrane. Water is a co-product of oxidation in the anode membrane. Hydrogen may also be produced from the dehydrogenation and coupling of hydrocarbons in the oxidation membrane of the reactor. The preferred temperature for reaction in the oxidation membrane is 400° to 1,000° C.

Reactor membranes useful for the present invention are prepared from materials that come from a family of cubic crystal lattice, A₆BO₈, wherein A and B are different elements and O is oxygen. These materials are solid solutions of B in A and have been observed to show very little crystal lattice parameter change upon reduction or re-oxidation, thus making them dimensionally stable as MIEC catalysts. Examples of materials include Mg₆MnO₈, Cu₆PbO₈ and Ni₆MnO₈, with Mg₆MnO₈ being particularly preferred.

In addition to stable crystal structures upon redox cycles, these A₆BO₈ materials have demonstrated high mixed ionic and electronic conductivity (MIEC) even at temperatures as low as room temperature making them particularly well-suited as materials used in the oxidation membrane in various embodiments of the present invention. In addition to the beneficial catalytic, and conductive, behavior in the class of the A₆BO₈ materials, they can be prepared in a way that yields very dense, hard, substrates. The addition of small amounts of boron greatly increases their particle toughness.

In addition to the A₆BO₈ materials, effective materials for selective oxidation for use in the present invention, particularly for the conversion of methane to higher hydrocarbons, preferably include at least one MIEC metal oxide that when contacted with a compound at the preferred conditions oxidizes the compound to a more unsaturated state or couples carbon-carbon bonds with the formation of water and at least one alkali metal or compound thereof. A more preferred composition will additionally include at least one of boron and compounds thereof. A most preferred composition comprises at least one MIEC oxide derived from any form of manganese oxide, manganese salt, or manganese compound, at least one alkali metal, alkaline earth metal, or compound thereof, at least one of boron and compounds thereof, and at least one oxide of alkaline earth metals. For example, the materials for use in the oxidation membrane may comprise an oxide of Mn, lithium (Li), boron (B), and manganese (Mg). The catalyst in the oxidation membrane may also contain one, or mixtures of, NaB₂Mg₄Mn₂O₄, NaB₂Mn₂Mg₄O_{11.5}, NaMn₂O₄, LiMn₂O₄, Mg₃Mn₃B₂O₁₀, Mg₃(BO₃)₂, or non-crystalline mixtures of these elements. Exemplary oxidation membrane materials that may be incorporated in various embodiments of the present invention are disclosed in U.S. Pat. Nos. 4,443,649; 4,444,984; 4,443,648; 4,443,645; 4,443,647; 4,443,644; 4,443,646; 4,499,324; 4,499,323; and 4,777,313, the contents of all of which are incorporated herein by reference.

The MIEC properties, i.e. electronic and ionic oxygen mobility, of the materials for use in the present invention may be enhanced by adding additional components and/or activators to the materials mentioned above or adding activators to the feed streams or oxygen fed to the oxidation or reduction membranes. Additional components include metal oxides selected from the group consisting of manganese (Mn), tin (Sn), indium (In), germanium (Ge), antimony (Sb), lead (Pb), bismuth (Bi), praseodymium (Pr), terbium (Tb), cerium (Ce), iron (Fe), ruthenium (Ru) and mixtures thereof. Examples of activators include silicates or aluminates of alkaline metals or alkaline earth metals, such as silicates and aluminates of sodium, lithium, calcium, and barium. In addition, silicates and aluminates of manganese (braunite), iron, zirconium, copper or ruthenium may be used. Another class of oxygen flux promoters that may be used includes oxides with hole structures that promote oxygen anion transport such as cerium oxide, zinc oxide, zirconium oxide (with or without additives such as yttrium), praseodymium oxide, or barium oxide. Gaseous activators that may be used include water, halogens, hydrogen sulfide, oxides of nitrogen, or any other material that aids in the activity and reactive lifetime of the catalyst.

In addition to increasing oxygen flux, if it is desired to increase the electronic conductivity of the materials, metals that have the ability to have multiple oxidation states in the temperature range of use may be added, such as ruthenium, copper, cobalt, iron, platinum, palladium, rhodium or chromium. The activators increase the rate of oxygen flux and electronic conductivity of the catalyst, thereby causing an increase in rate of selective oxidative conversion. The catalysts thus formed will be more active for the OCM to olefins and the oxidative dehydrogenation of hydrocarbons to olefins.

The catalysts so described in this invention are conveniently prepared by any methods known by those skilled in the art which include precipitation, co-precipitation, impregnation, granulation, spray drying, dry mixing or others. The catalyst precursors are transformed to the active catalysts by calcination at temperatures suitable for the formation of the active components, typically in the range of 400° to 1,100° C. The calcination may be performed under any atmosphere, such as air, inert gases, hydrogen, carbon monoxide, hydrocarbon gases so as to form the active catalyst composition.

The oxidation membrane may be produced in any method known by those skilled in the art of the production of solid oxide membrane reactors, such as an SOFC. The oxidation membrane may in the form of a catalytic membrane made by use of tape casting, plasma spray, screen printing, chemical vapor deposition, extrusion, sintering or any other known method.

The effluent produced by the reactor of the present invention may comprise unconverted methane and higher hydrocarbons as well as carbon oxides and water. It is within the scope of the present invention to recycle the effluent to the oxidation membrane prior to conducting the effluent to a vessel in which the intermediates are converted to a product. Similarly, carbon oxides and water may be removed from the effluent prior to further treatment. Whether or not such separations are employed, intermediates comprising an oxidized hydrocarbon stream containing olefins and other forms of unsaturation are generated by the reactor and all or a portion of such stream is passed to the second stage of the process of this invention wherein higher molecular weight products are produced by oligomerization as illustrated in FIG. 3. Numerous catalysts and processes are known for the oligomerization of olefins generally, and of ethylene particularly, all of which may be employed in the vessel (32). For example, phosphoric acid supported on a kieselguhr base has been widely used for making polymer gasoline (i.e., olefinic hydrocarbon liquids within the gasoline boiling range) from refinery gases. Other catalysts which have been employed for similar purposes include the oxides of cobalt, nickel, chromium, molybdenum and tungsten on supports such as alumina, silica-alumina, kieselguhr, carbon and the like.

Higher hydrocarbon products of interest may include aviation fuels, kerosene or intermediate refining streams.

Without intending to limit the scope of the claimed invention, most oligomerization catalysts may be classified in one of two general categories: metal catalysts and acid catalysts. They may also be classified as heterogeneous (solid) catalysts or homogeneous (liquid-phase) catalysts. Examples of metal catalysts that may be used in the vessel of the present invention for oligomerization of intermediates, include nickel (note that these catalysts require a donor ligand and a Lewis acid), palladium, chromium, cobalt, titanium, tungsten, and rhenium. Examples of acid catalysts include phosphoric acid and acid catalysts based on alumina.

Other acid catalysts that may be used in the present invention are silaceous, crystalline molecular sieves. Such silica-containing crystalline materials include materials which contain, in addition to silica, significant amounts of alumina, and generally known as “zeolites”, i.e., crystalline aluminosilicates. Silica-containing crystalline materials also include essentially aluminum-free silicates. These crystalline materials are exemplified by crystalline silica polymorphs (e.g., silicalite and organosilicates), chromia silicates (e.g., CZM), ferrosilicates and galliosilicates, and borosilicates. Crystalline aluminosilicate zeolites are best exemplified by ZSM-5, ZSM-11, ZSM12, ZSM-21, ZSM-38, ZSM-23, and ZSM-35.

Metal oligomerization catalysts in general are more sensitive to feed impurities, (e.g., water, carbon monoxide, dienes, etc.) than are the acid catalysts. Although homogeneous, metal catalysts are quite active, the need for dry feeds, solvents, and other measures to prevent catalyst deactivation and precipitation is disadvantageous and suggests an obvious advantage to supported, heterogeneous, metal catalyst.

Homogeneous acid catalysts are effective but are also corrosive and tend to form two liquid-phase systems with the non-polar hydrocarbon oligomerization products. Considering the foregoing observations, heterogeneous acid catalysts are the preferred catalyst for use in the oligomerization step of the present invention. Of the heterogeneous acid catalysts, acid zeolites are especially preferred, particularly zeolites of the ZSM-type and borosilicates.

### EXAMPLES

In order that the invention may be more fully understood, the following Examples are provided by way of illustration only.

### Example 1 (Working Example)

An anode catalyst was prepared by mixing 42.3 g of MgO, 32.3 g of MnO₂, 11.3 g of H₃BO₃, and 4.5 g of LiOH in sufficient deionized water to make a thick slurry. After thoroughly mixing the slurry mixture in a rotating ball mill for 2 hours, the resulting mixture was dried in air for 12 hours at 110° C. Once dried, the dry composition was heated in a furnace, in air, from room temperature to 1,000° C. at a rate of 10° C. per minute and held at 1,000° C. for 16 hours. The resulting catalyst was compressed into a cylindrical pellet of approximately 2 mm diameter and 2.5 mm length using a hydraulic press at 30,000 psi, and the pellet was analyzed by AC Impedance Spectroscopy using an Autolab potentiostat from 1 to 1,000 Hz, in air, at temperatures between 750° and 850° C. The AC conductivity was determined from the high frequency range of the spectrum and the DC impedance was interpolated from the low frequency range. The results (shown in Table 2) were compared to known average values for a typical 8% YSZ ionic conducting electrolyte material. The sample anode catalyst demonstrated MIEC properties and had a total conductivity similar to the electrolyte material, YSZ.

TABLE 2
Conductivities of Catalytic Membranes (S/cm) Measured by AC
Impedance Spectroscopy

| | | Example 1 | |
|---|---|---|---|
| | | Catalyst | YSZ |
| Total AC Conductivity | 750° C. | 0.019 | 0.02 |
| (Frequency 1 Hz-1 MHz) | 850° C. | 0.041 | 0.07 |
| DC Conductivity | 750° C. | 0.018 | NA |
| | 850° C. | 0.021 | NA |

### Example 2 (Working Example)

A sample of catalyst from Example 1 was placed into a micro-fixed bed reactor and produced the following activity and selectivity for the conversion of methane to higher hydrocarbons in Table 3. The sample designated as MIEC^{A} is the anode material. The methane conversion observed was in a “redox” mode, which means that methane was converted over the catalyst in the absence of air. In a separate step, the catalyst was re-activated and re-oxidized with air. The activity for methane conversion in the absence of air demonstrates that this catalyst functions to store oxygen in its structure and performs as an MIEC material.

TABLE 3
Catalytic OCM conversions with anode catalyst

| Cycle Length, | % Methane | % C₂⁺ | % C₂⁺ |
|---|---|---|---|
| Sec. | Conversion | Selectivity | Yield |
| 60 | 58.7 | 58.7 | 26.7 |

Conditions: MIEC^{A} catalyst from Example 1; 850° C., WHSV = 1/hr, average over 5 redox cycles.

### Example 3 (Working Example)

A sample of catalyst from Example 1 was tested in a button SOFC test stand with a configuration similar to FIG. 1. The membrane electrode assembly was composed of a 32 mm diameter, 300 μm thick, 8-YZS electrolyte onto which a 50 μm lanthanum-strontium-manganite cathode layer was applied. The anode surface was composed of a 50/50 by weight mixture of anode catalyst from Example 1 and 8-YSZ nano-particles which had been screen printed to approximately a 50 μm thickness. The total electrode working area was 1.25 cm². Electrical contacts on both the anode and cathode were made via a silver paste and mesh. When methane was introduced to the anode chamber and air to the cathode chamber, both at 900° C., the open circuit potential showed an induction period of about one hour, eventually stabilizing to about 0.8 V, as shown in FIG. 4 and as predicted by Table 1.

### Example 4 (Working Example)

An anode catalyst was prepared by impregnating MgO with 12.5% NaMnO₄ and calcining at 825° C. in air for 12 hours. A membrane electrode assembly was prepared with this catalyst using the same procedure as in Example 3. When methane was introduced to the anode chamber and air to the cathode chamber, the system functioned as a fuel cell, as shown in FIG. 1, and produced the polarization curve shown in FIG. 5. The introduction of 20 volume percent steam to the methane feed demonstrated an increase in voltage at lower cell current densities.

### Example 5 (Working Example)

An anode catalyst and membrane electrode assembly was prepared as in Example 4. Methane was introduced to the anode chamber and air to the cathode chamber. In this example current was applied to the cell, similar to the configuration in FIG. 3, over a range from zero (OCP) to 800 mA/cm². The methane conversion ranged from 0.75% at 50 mA/cm² (65% C₂⁺ selectivity) to approximately 2.5% at 800 mA/cm² (25% C₂⁺ selectivity). The principle non-selective products were carbon dioxide and carbon monoxide. The cell demonstrated stable electrochemical activity for over two hours. The anode catalyst activity was over 0.87 g methane converted per g-catalyst per hour. This is a turn-over rate greater than 3.5 times the conversion rate observed for this catalyst in fixed bed redox OCM runs.

### Example 6 (Working Example)

Process simulations were performed on reactor configurations similar to those shown in FIGS. 1 and 2 within a system in FIG. 3 and compared to process simulations for a methane to liquid plant modeled on a known Fischer-Tropsch gas to liquid system. The systems were simulated at a scale of 500 BL/D of gasoline product. The Fischer-Tropsch reactor system was assumed to use the best available technology for the production of synthesis gas via an oxygen fed partial oxidation unit. The overall product yields for the Fischer-Tropsch plant were assumed to be similar to an existing plant which generates naphtha, n-paraffins, kerosene, gas oil, and base oil. In contrast, the olefin oligomerization product from the reaction scheme was assumed to be a narrow boiling range gasoline. This example demonstrated that the electrogenerative reactor system, as described in the present invention, provided higher overall thermal efficiency than conventional Fischer-Tropsch GTL. In addition, the example demonstrated that the system similar to the embodiment illustrated in FIG. 2 can produce 500 BL/D of product from approximately 3,000 SCFC of methane feed with the concomitant direct production of 5 MW of electrical power. The higher efficiency for the process in FIG. 2 versus that of FIG. 1 and the Fischer-Tropsch process is a direct result of the higher efficiency of the electrogenerative reactor system to convert excess energy of the reaction when compared to conventional steam turbine systems, as used in the other two examples.

TABLE 4

| GTL Process | Overall Thermal Efficiency, % |
|---|---|
| As in FIG. 2 | 56-58 |
| Fischer-Tropsch GTL | 60-62 |
| As in FIG. 1 | 72-75 |

While preferred embodiments of the invention have been shown and described herein, it will be understood that such embodiments are provided by way of example only. Numerous variations, changes, and substitutions will occur to those skilled in the art without departing from the spirit of the invention. Accordingly, it is intended that the appended claims cover all such variations as fall within the spirit and scope of the invention.

What is claimed:

1. A reactor for the production of unsaturated hydrocarbons, said reactor comprising:
a solid oxidation membrane having MIEC properties, the oxidation membrane comprising a material having a cubic crystal lattice structure and a chemical formula of:
A₆BO₈
wherein A is a first element, B is a second element that is different than the first element, and O is oxygen, and
wherein the oxidation membrane includes an oxidation zone configured to receive a gaseous feedstream and oxidize components of the feedstream.

2. The reactor of claim 1, wherein the material is selected from the group consisting of Mg₆MnO₈, Cu₆PbO₈, and Ni₆MnO₈.

3. The reactor of claim 1, wherein the material is Mg₆MnO₈.

4. The reactor of claim 1, wherein the oxidation membrane further comprises at least one alkali metal.

5. The reactor of claim 4, wherein the oxidation membrane further comprises boron.

6. The reactor of claim 5, wherein the oxidation membrane further comprises an oxide of an alkaline earth metal.

7. The reactor of claim 1, wherein the oxidation membrane further comprises a metal oxide, the metal selected from the group consisting of tin, indium, germanium, antimony, lead, bismuth, praseodymium, zinc, zirconium, barium, and cerium.

8. The reactor of claim 1, wherein the oxidation membrane further comprises at least one of NaB₂Mg₄Mn₂O₄, NaB₂Mn₂Mg₄O_{11.5}, NaMn₂O₄, LiMn₂O₄, Mg₃Mn₃B₂O₁₀, Mg₃(BO₃)₂, and a non-crystalline mixture of compounds comprising oxygen and at least one of sodium, boron, magnesium, manganese, and lithium.

9. The reactor of claim 1, wherein the oxidation membrane further comprises a reduction zone configured to receive a gas containing oxygen and reduce the oxygen to an ionic form.

10. The reactor of claim 1 further comprising:
a solid reduction membrane having MIEC properties; and
an electron barrier between the oxidation membrane and the reduction membrane; and
a conductor attached to the oxidation membrane and the reduction membrane and configured to conduct electrons from the oxidation membrane to the reduction membrane,
wherein the reduction membrane is configured to receive a gas containing oxygen and reduce the oxygen to anionic oxygen, and
wherein the electron barrier is configured to allow transmission of the anionic oxygen from the reduction membrane to the oxidation membrane and resist transmission of electrons from the oxidation membrane to the reduction membrane.

11. The reactor of claim 10 wherein the electron barrier is configured to resist the passage of monovalent or molecular oxygen from the reduction membrane to the oxidation membrane.

12. The reactor of claim 10, wherein the conductor is configured to at least one of receive electrical power or allow electrical power to be withdrawn from the reactor.

13. A process for the production of a product comprising:
providing at least one of a hydrocarbon, sulfur containing compound, nitrogen containing compound, alcohol, and carbon monoxide to the oxidation membrane of a reactor according to claim 10;
supplying oxygen to the reduction membrane;
conducting an effluent containing an intermediate from the reactor to a vessel; and
converting the intermediate to a product in the vessel, wherein the product has a higher molecular weight than the intermediate.

14. The process according to claim 13, wherein the product is a liquid hydrocarbon.

15. The process according to claim 13, wherein the intermediate is converted in the vessel by oligomerization.

16. The process according to claim 13, further comprising:
a second conducting step to conduct the product and at least one of uncoverted intermediates, under-converted intermediates, carbon monoxide, and carbon dioxide from the vessel to a separator; and
separating the product in the separator from the at least one of uncoverted intermediates, under-converted intermediates, carbon monoxide, and carbon dioxide.

17. The process according to claim 16, further comprising recycling at least one of the unconverted intermediates and under-converted intermediates to the reactor.

18. The process according to claim 13 further comprising withdrawing electrical power from the conductor.

19. The process according to claim 13 further comprising applying electrical power to the conductor to increase oxygen ion conductivity through the electron barrier of the reactor.

20. The process according to claim 13, wherein the intermediates are oxygenated hydrocarbons, the effluent further comprises at least one of carbon monoxide and carbon dioxide, and the oxygenated hydrocarbons are present in the effluent at a higher molar concentration than at least one of the carbon monoxide and carbon dioxide.

21. A system for the production of a product from a gaseous feed, said system comprising:
a first feed stream comprising at least one of hydrogen, a hydrocarbon, a sulfur containing compound, a nitrogen containing compound, alcohol, and carbon monoxide;
a second feed stream comprising oxygen; and
a reactor configured to receive the first and second streams, operate within a temperature range, and produce an effluent, said reactor comprising a solid oxidation membrane having MIEC properties, the oxidation membrane comprising a material having a cubic crystal lattice structure and a chemical formula of:
A₆BO₈
wherein A is a first element, B is a second element that is different than the first element, and O is oxygen.

22. The system of claim 21, further comprising a vessel that receives the effluent and produces a product from an intermediate in the effluent, the product having a higher molecular weight than the intermediate.

23. The system of claim 21, further comprising a gaseous activator wherein the gaseous activator is combined with at least one of the first stream and the second stream and is selected from the group consisting of water, halogens, hydrogen sulfide, oxides of nitrogen, and mixtures thereof.

24. The system of claim 21, further comprising an electronic conductivity additive, wherein the electronic conductivity additive is combined with the MIEC oxide and is a metal having multiple oxidation states in the temperature range.
